(19) United States
(12) Patent Application Publication
(10) Pub. No.: US 2019/0119761 A1
(43) Pub. Date: Apr. 25, 2019

(54) GENES AND PERSONALISED TRAINING

(71) Applicant: DNAFIT LIFE SCIENCES LIMITED, London (GB)

(72) Inventors: Keith GRIMALDI, London (GB); Avi LASAROW, London (GB)

(73) Assignee: DNAFIT LIFE SCIENCES LIMITED, London (GB)

(21) Appl. No.: 16/089,741

(22) PCT Filed: Mar. 31, 2017
(86) PCT No.: PCT/GB2017/050913

§ 371 Date: Sep. 28, 2018

(30) Foreign Application Priority Data
Mar. 31, 2016 (GB) 1605417.3

(51) Int. Cl. C12Q 1/6888; C12Q 1/6827

(52) U.S. Cl. CPC C12Q 1/6888; C12Q 2600/156; C12Q 2600/124; C12Q 1/6827

(57) ABSTRACT

The invention relates to methods for identifying whether an individual has predominantly a power or endurance profile. In particular, it relates to methods for identifying a predisposition to an ability to respond well to high intensity or low-intensity resistance training by identifying the allele present at the locus of one or more of genetic polymorphisms.

### FIELD OF THE INVENTION

The invention relates to methods for identifying whether an individual has predominantly a power or endurance profile. In particular, it relates to methods for identifying a predisposition to an ability to respond well to high intensity or low-intensity resistance training.

### BACKGROUND TO THE INVENTION

Resistance exercise training is now widely used to enhance general fitness and athletic success in many sporting disciplines including power, strength and endurance events [1, 2]. When properly performed and combined with adequate nutrition, resistance training leads to increases in strength, power, speed, muscle size, local muscular endurance, coordination, and flexibility and reductions in body fat and blood pressure [3].

The proper resistance exercise prescription involves manipulation of several variables specific to the targeted goals, such as intensity or load per repetition (i.e. percentage of one repetition maximum (1 RM)), volume (total number of sets and repetitions), training frequency, muscle action (concentric vs. eccentric), rest intervals between sets, repetition velocity and others [3, 4]. Based on these variables, resistance training can be categorized into two common types: low-intensity (˜30% of 1 RM and high repetitions) and high-intensity (˜70% of 1 RM and low repetitions) resistance training. Low-intensity resistance training is effective for increasing absolute local muscular endurance [5], explosive power [6, 7] and preferential hypertrophy of slow-twitch muscle fibres [8, 9], while more widely used high-intensity training (also known as classic strength training) leads to increases in absolute strength [3] and the hypertrophy of all types of muscle fibres [10, 11].

There is a large variability in both muscle size and strength gains in response to resistance training between individuals [4]. In a large study of 585 subjects, Hubal et al. [12] have shown that men and women exhibited wide ranges of strength gain (1 RM: 0 to +250%) and skeletal muscle hypertrophy (cross-sectional area: −2 to +59%) of the non-dominant arm in response to 12-wk classic resistance training, indicating that i) each individual has his/her own genetic limit in the muscle size and strength gains in response to classic strength training; ii) non- and low responders to classic resistance training should alter training variables to improve the anthropometric and physiological characteristics of their skeletal muscles. Indeed, there is a general consensus that resistance training programs need to be individualized based on individual goals, strengths and weaknesses (i.e. genetic potential for the development of physical qualities) in order to maximize the outcomes [3, 4, 12, 13].

Given that muscle fibre composition is a heritable (˜45% genetic) trait [14], its variability (e.g. range 5-90% for slow-twitch muscle fibres in the vastus lateralis muscle) may determine individual's potential to perform different types of resistance training. Accordingly, data show that type I muscle fibres have high resistance to fatigue and are thus suited for low-intensity resistance or aerobic (endurance) training, IIA fibres are better suited for medium-term anaerobic exercise, and type IIX fibres are adapted for high-intensity (power and strength) exercise [8, 13, 15]. It should be noted that although muscle fibre composition is an informative biomarker (there is no inter-conversion between fast- and slow-twitch muscle fibres), because of the invasiveness muscle biopsies cannot be used widely. Therefore, for exercise prescription purposes other prediction tests, such as genetic testing, should be developed.

Association studies have identified dozens of genetic variants linked to training responses and sport-related traits, such as strength, skeletal muscle mass, recovery ability and muscle fibre composition [16-19]. However, no intervention studies utilizing the idea of personalised training based on the genetic profile of athletes have been carried out. The inventors have identified gene polymorphisms that may be used to predict an individual's response to resistance training. Further, the inventors have created an algorithm to combine those polymorphisms to provide even more prediction of how an athlete will respond to a high- or low-intensity resistance training program, by predicting an athlete's potential for the development of power and endurance qualities. It is particularly surprising that it is possible to predict not only whether an athlete will respond well to training, but also to what type of training will give the best results.

### SUMMARY OF THE INVENTION

The invention provides a method for predicting an individual's response to resistance training. In particular, the invention provides a method for predicting whether an individual will respond more to high intensity training or to low intensity training.

The method of the invention comprises identifying the allele present at the locus of one or more of the genetic polymorphisms shown in table 1, in a sample obtained from the individual. In particular, the method may comprise identifying the allele present at the locus of at least two, at least three, at least four or at least five, at least six, at least seven, at least eight, at least nine, at least ten, at least eleven, at least twelve, at least thirteen, at least fourteen or at least all of the polymorphisms shown in table 1.

The method may further comprise determining whether the allele identified is an indicator of endurance performance or of power performance.

In the method of the invention, the presence of one or more power performance allele may be used to determine that the individual is more likely to respond to high intensity training. The presence of one or more endurance allele may be used to determine that the individual is more likely to respond to low intensity training.

Particularly when the allele is a power allele, the one or more polymorphisms in the method of the invention may comprise or consist of one or more of ACE, ACTN3, CRP rs1205, PPARGC1A and VDR. It may also include only those polymorphisms. In further embodiments of the invention when the allele is a power allele, the one or more polymorphisms may comprise or consist of any of the following combinations of polymorphisms:

### ACE, ACTN3;

### ACE, CRP;

### ACE, PPARGC1A;

### ACE, VDR;

### ACE, ACTN3, CRP;

### ACE, ACTN3, PPARGC1A;

### ACE, ACTN3, VDR;

### ACE, CRP, PPARGC1A,

### ACE, CRP, VDR;

### ACE, PPARGC1A, VDR;

### ACE, ACTN3, CRP, PPARGC1A;

### ACE, ACTN3, CRP, VDR;

### ACE, ACTN3, CRP, PPARGC1A, VDR;

### ACTN3, CRP;

### ACTN3, PPARGC1A;

### ACTN3, VDR;

### ACTN3, CRP, PPARGC1A;

### ACTN3, CRP, VDR;

### ACTN3, PPARGC1A, VDR;

### CRP, PPARGC1A;

### CRP, VDR;

### CRP, PPARGC1A, VDR; and PPARGC1A, VDR.

Particularly when the allele is an endurance allele, the one or more polymorphisms in the method of the invention may comprise or consist of one or more of ACE, ACTN3, CRP, and VDR. It may also consist of only those polymorphisms. In further embodiments of the invention when the allele is a power allele, the one or more polymorphisms may comprise or consist of any of the following combinations of polymorphisms:

### ACE, ACTN3;

### ACE, CRP;

### ACE, VDR;

### ACE, ACTN3, CRP;

### ACE, ACTN3, VDR;

### ACE, CRP, VDR;

### ACE, ACTN3, CRP, VDR;

### ACTN3, CRP;

### ACTN3, VDR;

### ACTN3, CRP, VDR; and

### CRP, VDR.

The one or more polymorphisms of the invention may alternatively or additionally comprise any one or more of TRHR, PPARA and IL6. These polymorphisms may be used individually or in conjunction with other polymorphisms. For example, the combinations of polymorphisms mentioned above may further comprise one or more of TRHR, PPARA and IL6. For example, the polymorphisms may comprise or consist of any of:

### TRHR, ACE, ACTN3;

### TRHR, ACE, CRP;

### TRHR, ACE, PPARGC1A;

### TRHR, ACE, VDR;

### TRHR, ACE, ACTN3, CRP;

### TRHR, ACE, ACTN3, PPARGC1A;

### TRHR, ACE, ACTN3, VDR;

### TRHR, ACE, CRP, PPARGC1A,

### TRHR, ACE, CRP, VDR;

### TRHR, ACE, PPARGC1A, VDR;

### TRHR, ACE, ACTN3, CRP, PPARGC1A;

### TRHR, ACE, ACTN3, CRP, VDR;

### TRHR, ACE, ACTN3, CRP, PPARGC1A, VDR;

### TRHR, ACTN3, CRP;

### TRHR, ACTN3, PPARGC1A;

### TRHR, ACTN3, VDR;

### TRHR, ACTN3, CRP, PPARGC1A;

### TRHR, ACTN3, CRP, VDR;

### TRHR, ACTN3, PPARGC1A, VDR;

### TRHR, CRP, PPARGC1A;

### TRHR, CRP, VDR;

### TRHR, CRP, PPARGC1A, VDR;

### TRHR, PPARGC1A, VDR

### TRHR, ACE, ACTN3;

### TRHR, ACE, CRP;

### TRHR, ACE, PPARGC1A;

### TRHR, ACE, VDR;

### TRHR, ACE, ACTN3, CRP;

### TRHR, ACE, ACTN3, PPARGC1A;

### TRHR, ACE, ACTN3, VDR;

### TRHR, ACE, CRP, PPARGC1A,

### TRHR, ACE, CRP, VDR;

### TRHR, ACE, PPARGC1A, VDR;

### TRHR, ACE, ACTN3, CRP, PPARGC1A;

### TRHR, ACE, ACTN3, CRP, VDR;

### TRHR, ACE, ACTN3, CRP, PPARGC1A, VDR;

### TRHR, ACTN3, CRP;

### TRHR, ACTN3, PPARGC1A;

### TRHR, ACTN3, VDR;

### TRHR, ACTN3, CRP, PPARGC1A;

### TRHR, ACTN3, CRP, VDR;

### TRHR, ACTN3, PPARGC1A, VDR;

### TRHR, CRP, PPARGC1A;

### TRHR, CRP, VDR;

### TRHR, CRP, PPARGC1A, VDR;

### TRHR, PPARGC1A, VDR

### PPARA, ACE, ACTN3;

### PPARA, ACE, CRP;

### PPARA, ACE, PPARGC1A;

### PPARA, ACE, VDR;

### PPARA, ACE, ACTN3, CRP;

### PPARA, ACE, ACTN3, PPARGC1A;

### PPARA, ACE, ACTN3, VDR;

### PPARA, ACE, CRP, PPARGC1A,

### PPARA, ACE, CRP, VDR;

### PPARA, ACE, PPARGC1A, VDR;

### PPARA, ACE, ACTN3, CRP, PPARGC1A;

### PPARA, ACE, ACTN3, CRP, VDR;

### PPARA, ACE, ACTN3, CRP, PPARGC1A, VDR;

### PPARA, ACTN3, CRP;

### PPARA, ACTN3, PPARGC1A;

### PPARA, ACTN3, VDR;

### PPARA, ACTN3, CRP, PPARGC1A;

### PPARA, ACTN3, CRP, VDR;

### PPARA, ACTN3, PPARGC1A, VDR;

### PPARA, CRP, PPARGC1A;

### PPARA, CRP, VDR;

### PPARA, CRP, PPARGC1A, VDR;

### PPARA, PPARGC1A, VDR

### IL6, ACE, ACTN3;

### IL6, ACE, CRP;

### IL6, ACE, PPARGC1A;

### IL6, ACE, VDR;

### IL6, ACE, ACTN3, CRP;

### IL6, ACE, ACTN3, PPARGC1A;

### IL6, ACE, ACTN3, VDR;

### IL6, ACE, CRP, PPARGC1A,

### IL6, ACE, CRP, VDR;

### IL6, ACE, PPARGC1A, VDR;

### IL6, ACE, ACTN3, CRP, PPARGC1A;

### IL6, ACE, ACTN3, CRP, VDR;

### IL6, ACE, ACTN3, CRP, PPARGC1A, VDR;

### IL6, ACTN3, CRP;

### IL6, ACTN3, PPARGC1A;

### IL6, ACTN3, VDR;

### IL6, ACTN3, CRP, PPARGC1A;

### IL6, ACTN3, CRP, VDR;

### IL6, ACTN3, PPARGC1A, VDR;

### IL6, CRP, PPARGC1A;

### IL6, CRP, VDR;

### IL6, CRP, PPARGC1A, VDR;

### IL6, PPARGC1A, VDR

### TRHR, PPARA, ACE, ACTN3;

### TRHR, PPARA, ACE, CRP;

### TRHR, PPARA, ACE, PPARGC1A;

### TRHR, PPARA, ACE, VDR;

### TRHR, PPARA, ACE, ACTN3, CRP;

### TRHR, PPARA, ACE, ACTN3, PPARGC1A;

### TRHR, PPARA, ACE, ACTN3, VDR;

### TRHR, PPARA, ACE, CRP, PPARGC1A,

### TRHR, PPARA, ACE, CRP, VDR;

### TRHR, PPARA, ACE, PPARGC1A, VDR;

### TRHR, PPARA, ACE, ACTN3, CRP, PPARGC1A;

### TRHR, PPARA, ACE, ACTN3, CRP, VDR;

### TRHR, PPARA, ACE, ACTN3, CRP, PPARGC1A, VDR;

### TRHR, PPARA, ACTN3, CRP;

### TRHR, PPARA, ACTN3, PPARGC1A;

### TRHR, PPARA, ACTN3, VDR;

### TRHR, PPARA, ACTN3, CRP, PPARGC1A;

### TRHR, PPARA, ACTN3, CRP, VDR;

### TRHR, PPARA, ACTN3, PPARGC1A, VDR;

### TRHR, PPARA, CRP, PPARGC1A;

### TRHR, PPARA, CRP, VDR;

### TRHR, PPARA, CRP, PPARGC1A, VDR;

### TRHR, PPARA, PPARGC1A, VDR

### TRHR, IL6, ACE, ACTN3;

### TRHR, IL6, ACE, CRP;

### TRHR, IL6, ACE, PPARGC1A;

### TRHR, IL6, ACE, VDR;

### TRHR, IL6, ACE, ACTN3, CRP;

### TRHR, IL6, ACE, ACTN3, PPARGC1A;

### TRHR, IL6, ACE, ACTN3, VDR;

### TRHR, IL6, ACE, CRP, PPARGC1A,

### TRHR, IL6, ACE, CRP, VDR;

### TRHR, IL6, ACE, PPARGC1A, VDR;

### TRHR, IL6, ACE, ACTN3, CRP, PPARGC1A;

### TRHR, IL6, ACE, ACTN3, CRP, VDR;

### TRHR, IL6, ACE, ACTN3, CRP, PPARGC1A, VDR;

### TRHR, IL6, ACTN3, CRP;

### TRHR, IL6, ACTN3, PPARGC1A;

### TRHR, IL6, ACTN3, VDR;

### TRHR, IL6, ACTN3, CRP, PPARGC1A;

### TRHR, IL6, ACTN3, CRP, VDR;

### TRHR, IL6, ACTN3, PPARGC1A, VDR;

### TRHR, IL6, CRP, PPARGC1A;

### TRHR, IL6, CRP, VDR;

### TRHR, IL6, CRP, PPARGC1A, VDR;

### TRHR, IL6, PPARGC1A, VDR

### PPARA, TRHR, IL6, ACE, ACTN3;

### PPARA, TRHR, IL6, ACE, CRP;

### PPARA, TRHR, IL6, ACE, PPARGC1A;

### PPARA, TRHR, IL6, ACE, VDR;

### PPARA, TRHR, IL6, ACE, ACTN3, CRP;

### PPARA, TRHR, IL6, ACE, ACTN3, PPARGC1A;

### PPARA, TRHR, IL6, ACE, ACTN3, VDR;

### PPARA, TRHR, IL6, ACE, CRP, PPARGC1A,

### PPARA, TRHR, IL6, ACE, CRP, VDR;

### PPARA, TRHR, IL6, ACE, PPARGC1A, VDR;

### PPARA, TRHR, IL6, ACE, ACTN3, CRP, PPARGC1A;

### PPARA, TRHR, IL6, ACE, ACTN3, CRP, VDR;

### PPARA, TRHR, IL6, ACE, ACTN3, CRP, PPARGC1A, VDR;

### PPARA, TRHR, IL6, ACTN3, CRP;

### PPARA, TRHR, IL6, ACTN3, PPARGC1A;

### PPARA, TRHR, IL6, ACTN3, VDR;

### PPARA, TRHR, IL6, ACTN3, CRP, PPARGC1A;

### PPARA, TRHR, IL6, ACTN3, CRP, VDR;

### PPARA, TRHR, IL6, ACTN3, PPARGC1A, VDR;

### PPARA, TRHR, IL6, CRP, PPARGC1A;

### PPARA, TRHR, IL6, CRP, VDR;

### PPARA, TRHR, IL6, CRP, PPARGC1A, VDR;

### PPARA, TRHR, IL6, PPARGC1A, VDR.

The one or more polymorphisms may comprise one or more other polymorphism, especially a polymorphism taken from table 1. For example, any of the above combinations may be used in conjunction with ADRB2 (either marker). Alternatively, any of the above combinations may be used in conjunction with AGT. Alternatively, any of the above combinations may be used in conjunction with BDKRB2. Alternatively, any of the above combinations may be used in conjunction with COL5A1. Alternatively, any of the above combinations may be used in conjunction with GABPB1. Alternatively, any of the above combinations may be used in conjunction with VEGFA.

The method of the invention may include the step of weighting one or more of the polymorphisms. In particular, the allele at any one or more of ACE, ACTN3, CRP, PPARGC1A and VDR, or any of the combinations mentioned above may be weighted more heavily than alleles at other loci. The presence of a power allele at any of ACE, ACTN3, CRP, PPARGC1A and VDR, or any of the combinations mentioned above may be more indicative of the likelihood of the individual to respond to high intensity training, than a power allele at any other locus. The presence of an endurance allele at any of ACE, ACTN3, CRP, and VDR, or any of the combinations mentioned above may be more indicative of the likelihood of the individual to respond to low intensity training, than an endurance allele at any other locus.

The ACE polymorphism may be weighted by a factor of between about 1 and about 4, about 2 and about 4, about 2 and about 3, or about 3 and about 4, when compared to any of the other polymorphisms present in table 1. It may particularly be weighted by between about 2 and 4 when compared with one of the other polymorphisms. Additionally or alternatively, it may be weighted by between about 2 and about 4, or about 3 and about 4 when compared with the weighting of any of ADRB2 (either marker), AGT, BDKRB2, COL5A1, GABPB1, IL6, PPARA, TRHR, VEGFA.

The ACTN3 polymorphism may be weighted by a factor of between about 1 and about 4, about 2 and about 4, about 2 and about 3, or about 3 and about 4, when compared to any of the other polymorphisms present in table 1. It may particularly be weighted by between about 2 and 4 when compared with one of the other polymorphisms. Additionally or alternatively, it may be weighted by between about 2 and about 4, or about 3 and about 4 when compared with the weighting of any of ADRB2 (either marker), AGT, BDKRB2, COL5A1, GABPB1, IL6, PPARA, TRHR, VEGFA.

The CRP polymorphism may be weighted by a factor of between about 1 and about 4, about 2 and about 4, about 2 and about 3, or about 3 and about 4, when compared to any of the other polymorphisms present in table 1. It may particularly be weighted by between about 2 and 4 when compared with one of the other polymorphisms. Additionally or alternatively, it may be weighted by between about 2 and about 4, or about 3 and about 4 when compared with the weighting of any of ADRB2 (either marker), AGT, BDKRB2, COL5A1, GABPB1, IL6, PPARA, TRHR, VEGFA.

The PPARGC1A polymorphism may be weighted by a factor of between about 1 and about 4, about 2 and about 4, about 2 and about 3, or about 3 and about 4, when compared to any of the other polymorphisms present in table 1. It may particularly be weighted by between about 2 and 4 when compared with one of the other polymorphisms. Additionally or alternatively, it may be weighted by between about 2 and about 4, or about 3 and about 4 when compared with the weighting of any of ADRB2 (either marker), AGT, BDKRB2, COL5A1, GABPB1, IL6, PPARA, TRHR, VEGFA.

The VDR polymorphism may be weighted by a factor of between about 1 and about 4, about 2 and about 4, about 2 and about 3, or about 3 and about 4, when compared to any of the other polymorphisms present in table 1. It may particularly be weighted by between about 2 and 4 when compared with one of the other polymorphisms. Additionally or alternatively, it may be weighted by between about 2 and about 4, or about 3 and about 4 when compared with the weighting of any of ADRB2 (either marker), AGT, BDKRB2, COL5A1, GABPB1, IL6, PPARA, TRHR, VEGFA.

Where the one or more polymorphism comprises one or more of ADRB2 (either marker), AGT, BDKRB2, COL5A1, GABPB1, IL6, PPARA, TRHR, VEGFA, the one or more of ADRB2 (either marker), AGT, BDKRB2, COL5A1, GABPB1, IL6, PPARA, TRHR, VEGFA may be weighted by between about 0.25 and 0.75 when compared with any of ACE, ACTN3, CRP, PPARGC1A and VDR, when present, or by between about 1 and 2 when compared with any other polymorphism.

The sample may be any appropriate sample obtained from the individual, particularly a sample containing DNA. For example, it may be a sample of bodily fluid, particularly saliva, blood or serum, or it could be a tissue sample.

The alleles may be identified using any appropriate method. Various methods of genotyping (PCR, Real-time PCR, sequencing, using micro-array, etc.) are known in the art.

The method of the invention allows a predisposition for response to a particular type of training to be identified. The method may include the step of testing the aerobic fitness or explosive power of the individual before and/or after training. Improved aerobic fitness and/or improved explosive power fitness may be used as indicators of response to training.

### DESCRIPTION OF THE DRAWINGS

The invention will now be described by way of example with reference to the following figures in which:

FIG. 1 is a graphical representation of the results of the power tests of Example 3 and shows that players who had trained on smaller pitches (power players doing power training) saw significantly greater improvements than players who had trained on larger pitches (endurance players doing endurance training).

FIG. 2 is a graphical representation of the results of the endurance tests of Example 3 and shows that players who had trained on larger pitches (endurance players doing endurance training) saw significantly greater improvements than players who had trained on smaller pitches (power players doing power training).

### DETAILED DESCRIPTION OF THE INVENTION

### Example 1

The inventors have identified 15 polymorphisms located within the genes primarily involved in the regulation of muscle fibre type composition and muscle size, cytoskeletal function, muscle damage protection, metabolism, circulatory homeostasis, mitochondrial biogenesis, thermogenesis and angiogenesis as being particularly useful in predicting an athlete's response to training. The inventors tested, in two independent studies, the hypothesis that genetically matched athletes (i.e. high-intensity trained with power genotype or low-intensity trained with endurance genotype) show greater improvements in explosive power (countermovement jump) and aerobic fitness (aerobic 3-min cycle test) in response to high- or low-intensity resistance training compared to mismatched athletes (i.e. high-intensity trained with endurance genotype or low-intensity trained with power genotype).

The inventors performed two studies in independent cohorts of male athletes (study 1: athletes from different sports (n=28); study 2: soccer players (n=39)). In both studies athletes completed an eight-week high- or low-intensity resistance training program, which either matched or mismatched their individual genotype. Two variables of explosive power and aerobic fitness, as measured by the countermovement jump (CMJ) and aerobic 3-min cycle test (Aero3) were assessed pre and post 8 weeks of resistance training. In study 1, the athletes from the matched group (i.e. high-intensity trained with power genotype or low-intensity trained with endurance genotype) significantly increased their results in CMJ (P=0.0005) and Aero3 (P=0.0004). On the other hand, athletes from the mismatched group (i.e. high-intensity trained with endurance genotype or low-intensity trained with power genotype) have shown non-significant improvements in CMJ (P=0.175) and less prominent results in Aero3 (P=0.0134). In study 2, soccer players from the matched group have also demonstrated significantly greater (P<0.0001) performance changes in both tests compared to mismatched group. Among non- or low responders of two studies, 82% of athletes (both for CMJ and Aero3) were from the mismatched group, while high responders were predominantly matched athletes (83% and 86% for CMJ and Aero3, respectively; P<0.0001). These results indicate that matching the individual's genotype with the appropriate training modality leads to more effective resistance training. The developed algorithm may be used to guide individualized resistance-training interventions.

### Methods

### Study Participants

In Study 1, 55 Caucasian male University athletes, all aged 18-20 years, volunteered for the study, and 28 of them (height 180.7±1.5 cm, weight 77.0±2.1 kg) successfully completed it (27 athletes had not completed all aspects of the study due to either injury or illness). Each participant was a member of first or second team, actively competing in British Universities and Colleges Sports (BUCS) leagues. The athletes competed in squash (n=1), swimming (n=7), running (n=1), ski/snowboard (n=4), soccer (n=1), lacrosse (n=2), badminton (n=1), motorsport (n=1), cycling (n=4), cricket (n=2), volleyball (n=1), fencing (n=1) and rugby union (n=2).

In study 2, 68 male soccer players, all aged 16-19 years, volunteered to participate in the study, and 39 of them (height 176.1±1.0 cm, weight 68.9±1.5 kg) successfully completed it (29 participants were withdrawn from the study due to non-adherence of set training volumes over the 8 weeks, or injury. The exclusions, in both studies, happened before the data was examined). Each subject was a member of college soccer academy who actively competed in BUCS leagues.

### Study Design

Study design utilised a time series trial as explained by Batterham and Hopkins [45]. Participants of both studies were randomly allocated to an eight-week high- or low-intensity resistance-training program, after undergoing performance tests for both explosive power and endurance. Participants transitioned from their normal training plan to the designed 8-week intervention followed by an eight-week wash-out period. The study was double blinded, in that all were unaware of their ‘genetic potential status’, as determined by the method of the invention. This also included the lead investigator who coached the participants during the 8 weeks of resistance training.

Prior to involvement in the study, all participants had undertaken weekly strength and conditioning programs, supervised by an accredited strength and conditioning coach, for a minimum of six months and maximum of two and half years. These sessions took place in a freeweights facility where technique and adherence was closely monitored at all times. Participants engaged in a minimum of one, and maximum of two (preferentially), sessions per week. No other form of resistance training was undertaken during this time, and participants were actively partaking in other sport-specific training sessions and competitive games in parallel to the intervention. The investigator selected the same exercises for both groups: deadlift, pulldowns, front squat to 90 degrees, dumbbell flat press, step ups to medium high box and vertical jump single effort.

Each group self-selected training loads for each session, were monitored for progressive increases in perceived exertion, using a modified Borg scale, and loads were recorded to ensure progression. The only differences between the training programs were volume modifications. The high-intensity resistance training program consisted of ten sets of two reps over the eight-week study. This gave a total volume of one hundred and twenty reps per session. The low-intensity resistance training program consisted of three sets of ten reps for first two weeks, three sets of fifteens reps for the next three weeks and three sets of twenty for the last three weeks. This gave a total volume of one hundred and eighty reps in the first two weeks, two hundred and seventy in the next three weeks and three hundred and sixty reps in the last three weeks.

### Physiological Measurements

All participants undertook a pre- and post-test measure of explosive power and aerobic fitness (endurance performance); namely, a countermovement jump (CMJ) and Aerobic 3-min Cycle test (Aero3), using a Optojump (Microgate, Italia) and Wattbike Pro (Wattbike, Nottingham, UK), respectively. Participants performed a standardized warm up before every testing session with the CMJ preceding the Aero3. Subjects were requested to arrive for testing in a rested and hydrated state and to refrain from caffeine intake for at least 12 hours before testing. Testing took place on the same time and weekday on each occasion, to ensure a consistent placement within the subject's usual schedule.

### Genotyping

Upon enrollment into study each participant volunteered a saliva sample, which was collected through sterile and self-administered buccal swabs. Samples were sent to IDna Genetics laboratory (Norwich, UK) within thirty-six hours, where analysis of the genes detailed in Table 1 was undertaken. DNA was extracted and purified using the Isohelix Buccalyse DNA extraction kit BEK-50 (Kent, UK). DNA samples were amplified by real-time PCR on an AB17900 real-time thermocycler (Applied Biosystem, Waltham, USA).

### Calculation of Power/Endurance Ratio

Following the analysis, each individual was given a percentage power/endurance score (P/E) ratio, similar to the research conducted by Egorova et al. [46]. Initially, each allele was given a point (0, 1, 2, 3 or 4) depending on the effect of the polymorphism on performance (power/muscle hypertrophy or endurance with respect to response to training). The total points for the P/E were expressed as a percentage of P/E and then combined to give the balance percentage. A percentage-ranking list was then complied using this score. Every other participant on the list then undertook high- or low-intensity resistance training. To clarify, someone who is 75% power but does low-intensity resistance training would be doing mismatched genotype training, while a participant rated as 75% endurance that completed low-intensity resistance training would be doing matched genotype training. A threshold for 50% was used as the splitting value in this process.

### Statistical Analysis

Statistical analysis was conducted in SPSS, Version 20 (Chicago, Ill.). The required sample size for this study was validated using the Mann-Whitney test. The chi-square test was used to test genotype distributions for deviation from Hardy-Weinberg equilibrium. The non-parametric 2-sample paired test was performed matching “before” and “after” measurements from each individual tested. A 2-sided Mann-Whitney test for 2 independent samples was used to compare gains in CMJ and Aero3 between groups. Differences in phenotypes between different genotype groups were analysed using ANOVA or unpaired t test. Spearman's (non-parametric) correlations were used to assess the relationships between the genotype score and performance tests. Bonferroni's correction for multiple testing was performed by multiplying the P value with the number of tests where appropriate. All data are presented as mean (standard deviation). Statistical significance was set at a P value<0.05. All data are shown as mean (SD).

### Results

### Efficiency of Different Training Modalities

All performance parameters increased significantly (<0.001) in response to low- and high-intensity resistance training when the results of two studies were combined. No significant differences in explosive power (CMJ: 5.4 (5.0) vs. 4.6 (6.1)%, P=0.547) and aerobic fitness (Aero3: 4.3 (3.8) vs. 4.3 (3.7)%, P=0.711) gains were observed between low- and high-intensity resistance training groups, indicating that i) both training modalities can be used to improve these performance parameters and ii) results of responses to both training types can be combined for the analysis where appropriate.

### Association Analysis Between Genotypes and Phenotypes

With some exceptions for the GABPB1 and VDR gene polymorphisms in Study 2 (due to the low sample sizes in terms of population genetics), genotype distributions of 15 gene polymorphisms amongst all athletes of both studies were in Hardy-Weinberg equilibrium (Table 2).

To assess the association between each polymorphism and performance parameters we used the combined data of two studies. After Bonferroni's correction for multiple testing the results were considered significant with P<0.0033 (i.e. 0.05/15). In accordance with the literature data (Table 1), we found that athletes with the ACE DD (P>0.1 for CMJ, P>0.1 for Aero3), ACTN3 Arg/Arg (P=0.065 for CMJ, P=0.0038 for Aero3), CRP rs1205 GG (P>0.1 for CMJ, P=0.0833 for Aero3), PPARGC1A Ser/Ser (P=0.065 for CMJ, P=0.0499 for Aero3) and VDR AA (P>0.1 for CMJ, P>0.1 for Aero3) genotypes demonstrated a tendency to have greater gains in one or two performance tests compared with the opposite genotype carriers after high-intensity resistance training, while the latter (except for the PPARGC1A polymorphism) better responded to the low-intensity training (ACE II: P>0.1 for CMJ, P=0.0355 for Aero3; ACTN3 Ter/Ter: P>0.1 for CMJ, P>0.1 for Aero3; CRP rs1205 AA: P=0.0224 for CMJ, P>0.1 for Aero3; VDR GG (P>0.1 for CMJ, P=0.0311 for Aero3). No significant differences in CMJ and Aero3 gains were observed between different genotype groups with respect to the other polymorphisms (data not shown). However, given that the latter 10 polymorphisms have recently been reported to be associated with endurance, power and muscle-specific traits, and the fact that each contributing gene can explain only a small portion of the observed interindividual differences in training-induced effects, we felt justified in retaining all 15 genetic markers for further analysis.

### Effect of Different Training Modalities and Genetic Profiles on Performance Parameters

Based on power/endurance genotype score (see Methods), in two studies we identified 39 athletes (58.2%) with endurance genotype and 28 athletes (41.8%) with power genotype profiles. Changes in CMJ and Aero3 tests of athletes with predominantly endurance or power genotype profiles from both studies after 8 weeks of low- and high-resistance training are presented in Tables 3 and 4. In both studies it was shown that athletes with endurance genotype profile had greater benefits from the low-intensity resistance training, while athletes with power genotype profile better responded to the high-intensity resistance training. As expected, the outcomes were more prominent in the Study 2 with homogeneous cohort (i.e. soccer players). Furthermore, we found that power genotype score (%) of athletes from both studies was positively correlated with CMJ (r=0.56; P=0.0005) and Aero3 (r=0.39; P=0.0199) increases (%) in response to high-intensity training, while endurance genotype score (%) was positively correlated with CMJ (r=0.37; P=0.0399) and Aero3 (r=0.51; P=0.0032) increases (%) in response to low-intensity training.

In accordance with power/endurance genotype score and training modality, 34 athletes performed matched training (high-intensity training with power genotype (n=15) or low-intensity training with endurance genotype (n=19)), while other 33 athletes completed mismatched training (high-intensity training with endurance genotype (n=20) or low-intensity training with power genotype (n=13)). In study 1, the athletes from the matched group have significantly increased their results in CMJ (P=0.0005) and Aero3 (P=0.0004). On the other hand, athletes from the mismatched group have shown non-significant improvements in CMJ (P=0.175) and less prominent results in Aero3 (P=0.0134) (Table 5). In study 2, soccer players from the matched group have also demonstrated significantly greater (P<0.0001) performance changes in both tests compared to mismatched group (Table 5).

### Determinants of Variability in Response to Resistance Training

With respect to the changes in CMJ gains (%), the athletes from both studies (n=67) were divided into tertiles: high responders (increase in CMJ from 7.4 to 19.4%; n=23), moderate responders (increase in CMJ from 2.7 to 7.2%; n=22) and non- or low responders (increase in CMJ from −8.4 to 2.5%; n=22). There was a significant linear trend for the proportion of matched-trained athletes among the high responders (82.6%), moderate responders (50.0%) and non- or low responders (18.2%) (χ²=18.7, P<0.0001). Similarly, when considering increases of Aero3(%), we found a significant linear trend for the proportion of matched-trained athletes among the high (increase in Aero3 from 6.0 to 13.2%; n=22) responders (86.4%), moderate (increase in Aero3 from 2.0 to 5.9%; n=23) responders (47.8%) and non- or low (increase in Aero3 from −6.1 to 1.9%; n=22) responders (18.2%) (χ²=20.5, P<0.0001). In other words, among non- or low responders to any type of resistance training, 82% of athletes (both for CMJ and Aero3) were from the mismatched group, while high responders were predominantly matched athletes (83% and 86% for CMJ and Aero3, respectively; P<0.0001 for the comparison between non- or low responders and high responders). Accordingly, after 8 weeks of resistance training the odds of achieving more favorable outcomes in CMJ and Aero3 were 21 and 28.5 times, respectively, greater (P<0.0001) for matched than mismatched genotype training (when first and third tertiles were compared).

### Discussion

To the best of our knowledge, this is the first study to examine the efficacy of using genetic profiling methods to target training of both power and endurance qualities of athletes. The results of our study demonstrated that all performance parameters increased significantly in response to 8-week low- or high-intensity resistance training without differences between two training modalities, however, these effects were dependent on the consistency between genetic profile and type of training. Our main finding is that matching the individual's genotype with the appropriate training modality leads to more effective resistance training, for both power and endurance matched participants. More specifically, in the first study we have shown that athletes from the matched group have significantly increased their results in explosive power and aerobic fitness, while mismatched athletes were less successful in these improvements. Importantly, these results were replicated in the second study of a homogenous cohort of athletes, and in combination with the first study these findings became more significant. There was also a positive correlation between power genotype score of athletes and performance changes in response to high-intensity training, as well as positive correlation between endurance genotype score and increases in both performance tests in response to low-intensity training, pointing to the fact that heterogeneity in resistance training-induced explosive power and aerobic fitness responses may be partly explained by genetic factors and selected training modalities. Another important finding of our study was that among non- or low responders to any type of resistance training, most athletes were from the mismatched group, while high responders were predominantly matched athletes. These results suggest that personalised training may help some individuals overcome unresponsiveness to resistance training.

Exercise training response is influenced by a multitude of determinants including genetics, environmental factors, measurement errors and others. Studies suggest that muscle strength and explosive power are under moderate to high genetic control with heritabilities ranging between 30 and 84% [17, 47]. Numerous studies reported the association between individual differences in strength/anaerobic power phenotypes in response to resistance/anaerobic power training and gene variations [16, 17]. Accordingly, several gene polymorphisms in our study were found to be individually linked with training responses. For instance, the II genotype of the ACE and XX (Ter/Ter) genotype of the ACTN3 genes (known as endurance markers) were associated (or tended to correlate) with increases in aerobic fitness in response to low-intensity resistance training, while the ACE DD and ACTN3 RR (Arg/Arg) genotypes (known as power/strength markers) carriers demonstrated greater improvement of performance parameters in response to high-intensity resistance training.

The likely mechanism through which the polygenic profile (i.e. profile composed of 15 polymorphisms) of athletes was associated with training responses could be the link between genetic variations and skeletal muscle characteristics, such as muscle fibre composition. Of note, 5 of 15 gene polymorphisms (ACE I/D, ACTN3 rs1815739 C/T, PPARA rs4253778 G/C, PPARGC1A rs8192678 G/A and VEGFA rs2010963 G/C) included in our panel, have recently been reported to be associated with muscle fibre type [18]. It is well known that slow-twitch muscle fibres better respond to low-intensity resistance or aerobic (endurance) training, while fast-twitch muscle fibres are better suited for high-intensity (power and strength) training [8, 13, 15]. Consequently, elite endurance athletes have a remarkably high proportion of slow-twitch muscle fibres, whereas muscles of top sprinters and weightlifters predominantly consist of fast-twitch muscle fibres [15]. Interestingly, Sukhova et al. [52] have shown that speed skaters whose muscle fibre composition did not correspond to their distance specialty (i.e. speed skaters with increased proportion of slow-twitch muscle fibres who performed sprint training and speed skaters with predominantly fast-twitch muscle fibres who performed endurance training) had destructive alterations of their muscles (with possible negative effect on physical performance), indicating that individuals should train and select sports in accordance with their genetic potential. One might speculate that non- or low-responders to different training modalities in our study genetically were not suited for selected resistance training types. On the other hand, there are many more factors at the molecular, cellular, tissue and organ system levels that may determine individual responses to resistance training. For instance, Petrella et al. [53] have demonstrated that extreme responders (in terms of hypertrophy of muscle fibres) to a 16-week resistance training program showed a markedly higher activation of their satellite cells and greater myonuclei addition compared with moderate responders and non-responders.

In conclusion, our results indicate that matching the individual's genotype with the appropriate training modality leads to more effective resistance training.

### Example 2

A study was conducted alongside Portsmouth College where genotype matching was used to monitor response to training in a group of collegiate soccer players. The players underwent three discrete training blocks:
1. Aerobic training,
2. Speed endurance training,
3. Sprint training.

No modifications of training interventions were made; instead the training response was monitored to see if the genotype groups saw different training adaptations. For the purpose of analysis, the athletes were split into “power” athletes (>50% power score) and “endurance” athletes (<50% power score). There were no athletes with a 50-50 split.

It was found that during the aerobic training block, endurance players saw greatest improvements in the Counter-Movement Jump (CMJ) test, improving by an average of just under 6%. In this same block, power players saw a decrement in CMJ performance. Aerobic training would be classed as “endurance-based” training; as such, genotype-matched players (endurance players doing endurance training) saw greater improvements in CMJ than genotype mismatched players (power players doing endurance training), as predicted by the Algorithm.

It was also found that during the sprint training block, power players saw greatest improvements in the CMJ, improving by almost 6%. In the same block, endurance players saw no improvement in CMJ. Sprint training would be classed as “power-based” training; as such, genotype-matched players (power players doing power training) saw greater improvements in CMJ than genotype mismatched players (endurance players doing power training), as predicted by the Algorithm.

Within this study, markers of mental well-being and mental toughness were also monitored. The results indicate that when training is matched to genotype (i.e., genetically matched training), mental toughness is improved. However, when training is mismatched to genotype (i.e. genetically mismatched training), players saw a reduction in mental toughness scores. This indicates that matched training, as determined by the algorithm, can improve mental toughness in individual.

### Example 3

Forty youth soccer players undertook eight weeks of sport-specific aerobic training in the form of small sided games. Training was matched to the individual genotype of the players as follows:
A. Endurance players: training on larger pitches, requiring longer duration running activities with a greater aerobic component.
B. Power players: training on smaller pitches, requiring a high number of short sprints with multiple accelerations representative of typical power-based training.

The players underwent pre- and post-training tests of power (CMJ and 10 m sprint) and endurance (maximum 3-minute cycle).

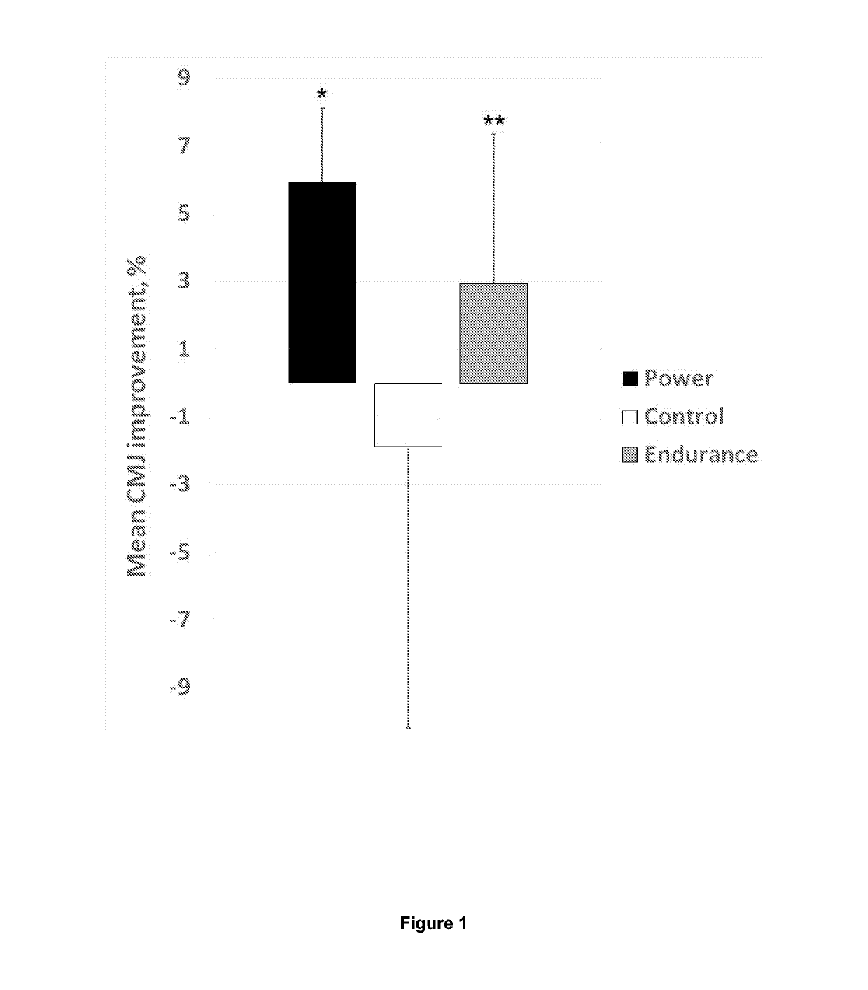

The results for the power tests are given in FIG. 1 showing that players who trained on smaller pitches (power players doing power training) saw significantly greater improvements than players who had trained on larger pitches (endurance players doing endurance training).

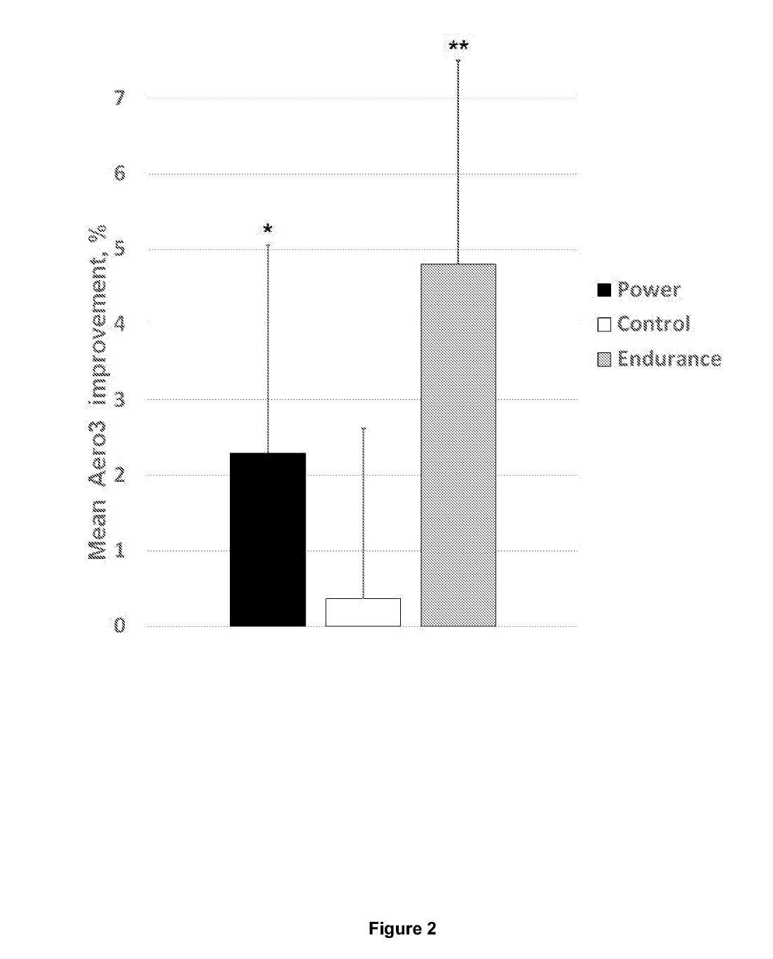

The results for the endurance tests are given in FIG. 2 showing that players who trained on larger pitches (endurance players doing endurance training) saw significantly greater improvements than players who had trained on smaller pitches (power players doing power training).

The combined results of Examples 2 and 3 further support the matching of an individual's genotype for the modification of training to improve fitness. The results also indicate that when players match their genotype to their training type, they see far greater improvements in fitness than players undertaking mismatched training types.

### REFERENCES

1. American College of Sports Medicine. American College of Sports Medicine position stand. Progression models in resistance training for healthy adults. Med Sci Sports Exerc. 2009; 41(3):687-708.
2. Vikmoen O, Ellefsen S, Trøen Ø, Hollan I, Hanestadhaugen M, Raastad T, Rønnestad B R. Strength training improves cycling performance, fractional utilization of VO2 max and cycling economy in female cyclists. Scand J Med Sci Sports. 2015 Apr. 18. doi: 10.1111/sms.12468.
3. Kraemer W J, Ratamess N A. Fundamentals of resistance training: progression and exercise prescription. Med Sci Sports Exerc. 2004; 36(4):674-688.
4. McGlory C, Phillips S M. Exercise and the Regulation of Skeletal Muscle Hypertrophy. Prog Mol Biol Transl Sci. 2015; 135:153-173.
5. Campos G E, Luecke T J, Wendeln H K, Toma K, Hagerman F C, Murray T F, Ragg K E, Ratamess N A, Kraemer W J, Staron R S. Muscular adaptations in response to three different resistance-training regimens: specificity of repetition maximum training zones. Eur J Appl Physiol. 2002; 88(1-2):50-60.
6. Wilson G J, Newton R U, Murphy A J, Humphries B J. The optimal training load for the development of dynamic athletic performance. Med Sci Sports Exerc. 1993; 25(11):1279-1286.
7. McBride J M, Triplett-McBride T, Davie A, Newton R U. The effect of heavy- vs. light-load jump squats on the development of strength, power, and speed. J Strength Cond Res. 2002; 16(1):75-82.
8. Netreba A I, Popov D V, Liubaeva E V, Bravyĭ Ia R, Prostova A B, Lemesheva Iu S, Vinogradova O L. Physiological effects of using the low intensity strength training without relaxation in single-joint and multi-joint movements. Ross Fiziol Zh Im I M Sechenova. 2007; 93(1):27-38.
9. Mitchell C J, Churchward-Venne T A, West D W, Burd N A, Breen L, Baker S K, Phillips S M. Resistance exercise load does not determine training-mediated hypertrophic gains in young men. J Appl Physiol (1985). 2012; 113(1):71-77.
10. Fry A C. The role of resistance exercise intensity on muscle fibre adaptations. Sports Med. 2004; 34(10):663-679.
11. Kosek D J, Kim J S, Petrella J K, Cross J M, Bamman M M. Efficacy of 3 days/wk resistance training on myofiber hypertrophy and myogenic mechanisms in young vs. older adults. J Appl Physiol (1985). 2006; 101(2):531-544.
12. Hubal M J, Gordish-Dressman H, Thompson P D, Price T B, Hoffman E P, Angelopoulos T J, Gordon P M, Moyna N M, Pescatello L S, Visich P S, Zoeller R F, Seip R L, Clarkson P M. Variability in muscle size and strength gain after unilateral resistance training. Med Sci Sports Exerc. 2005; 37(6):964-972.
13. Pipes T V. Strength training and fiber types. Scholastic Coach. 1994; 63:67-70.
14. Simoneau J-A, Bouchard C. Genetic determinism of fiber type proportion in human skeletal muscle. FASEB J. 1995; 9:1091-1095.
15. Andersen J L, Schjerling P, Saltin B. Muscle, genes, and athletic performance. Sci Am. 2000; 283(3):48-55.
16. Bray M S, Hagberg J M, Perusse L, Rankinen T, Roth S M, Wolfarth B, Bouchard C. The human gene map for performance and health-related fitness phenotypes: the 2006-2007 update. Med Sci Sports Exerc. 2009; 41(1):35-73.
17. Hughes D C, Day S H, Ahmetov I I, Williams A G. Genetics of muscle strength and power: polygenic profile similarity limits skeletal muscle performance. J Sports Sci. 2011; 29(13):1425-34.
18. Ahmetov I I, Vinogradova O L, Williams A G. Gene polymorphisms and fiber-type composition of human skeletal muscle. Int J Sport Nutr Exerc Metab. 2012; 22(4):292-303.
19. Ahmetov I I, Fedotovskaya O N. Current Progress in Sports Genomics. Adv Clin Chem. 2015; 70:247-314.
20. Ma F, Yang Y, Li X, Zhou F, Gao C, Li M, Gao L. The association of sport performance with ACE and ACTN3 genetic polymorphisms: a systematic review and meta-analysis. PLoS One. 2013; 8(1):e54685.
21. Wang G, Mikami E, Chiu L L, D E Perini A, Deason M, Fuku N, Miyachi M, Kaneoka K, Murakami H, Tanaka M, Hsieh L L, Hsieh S S, Caporossi D, Pigozzi F, Hilley A, Lee R, Galloway S D, Gulbin J, Rogozkin V A, Ahmetov I I, Yang N, North K N, Ploutarhos S, Montgomery H E, Bailey M E, Pitsiladis Y P. Association analysis of ACE and ACTN3 in elite Caucasian and East Asian swimmers. Med Sci Sports Exerc. 2013; 45(5):892-900.
22. Yang N, Arthur D G, Gulbin J P, Hahn A G, Beggs A H, Easteal S, North K. ACTN3 genotype is associated with human elite athletic performance. Am J Hum Genet. 2003; 73(3):627-631.
23. Wolfarth B, Rankinen T, Mühlbauer S, Scherr J, Boulay M R, Perusse L, Rauramaa R, Bouchard C. Association between a beta2-adrenergic receptor polymorphism and elite endurance performance. Metabolism. 2007; 56(12):1649-1651.
24. Tsianos G I, Evangelou E, Boot A, Zillikens M C, van Meurs J B, Uitterlinden A G, Ioannidis J P. Associations of polymorphisms of eight muscle- or metabolism-related genes with performance in Mount Olympus marathon runners. J Appl Physiol (1985). 2010; 108(3):567-574.
25. McCole S D, Shuldiner A R, Brown M D, Moore G E, Ferrell R E, Wilund K R, Huberty A, Douglass L W, Hagberg J M. Beta2- and beta3-adrenergic receptor polymorphisms and exercise hemodynamics in postmenopausal women. J Appl Physiol (1985). 2004; 96(2):526-530.
26. Gomez-Gallego F, Santiago C, Gonzalez-Freire M, Yvert T, Muniesa C A, Serratosa L, Altmae S, Ruiz J R, Lucia A. The C allele of the AGT Met235Thr polymorphism is associated with power sports performance. Appl Physiol Nutr Metab. 2009; 34(6):1108-1111.
27. Zarȩbska A, Sawczyn S, Kaczmarczyk M, Ficek K, Maciejewska-Karlowska A, Sawczuk M, Leońska-Duniec A, Eider J, Grenda A, Ciȩszczyk P. Association of rs699 (M235T) polymorphism in the AGT gene with power but not endurance athlete status. J Strength Cond Res. 2013; 27(10):2898-2903.
28. Posthumus M, Schwellnus M P, Collins M. The COL5A1 gene: a novel marker of endurance running performance. Med Sci Sports Exerc. 2011; 43(4):584-589.
29. Brown J C, Miller C J, Posthumus M, Schwellnus M P, Collins M. The COL5A1 gene, ultra-marathon running performance, and range of motion. Int J Sports Physiol Perform. 2011; 6(4):485-496.
30. Obisesan T O, Leeuwenburgh C, Phillips T, Ferrell R E, Phares D A, Prior S J, Hagberg J M. C-reactive protein genotypes affect baseline, but not exercise training-induced changes, in C-reactive protein levels. Arterioscler Thromb Vasc Biol. 2004; 24(10):1874-1879.
31. Kuo H K, Yen C J, Chen J H, Yu Y H, Bean J F. Association of cardiorespiratory fitness and levels of C-reactive protein: data from the National Health and Nutrition Examination Survey 1999-2002. Int J Cardiol. 2007; 114(1):28-33.
32. He Z, Hu Y, Feng L, Lu Y, Liu G, Xi Y, Wen L, McNaughton L R. NRF2 genotype improves endurance capacity in response to training. Int J Sports Med. 2007; 28(9):717-721.
33. Eynon N, Sagiv M, Meckel Y, Duarte J A, Alves A J, Yamin C, Sagiv M, Goldhammer E, Oliveira J. NRF2 intron 3 A/G polymorphism is associated with endurance athletes' status. J Appl Physiol (1985). 2009; 107(1):76-79.
34. Ruiz J R, Buxens A, Artieda M, Arteta D, Santiago C, Rodriguez-Romo G, Lao J I, Gomez-Gallego F, Lucia A. The −174 G/C polymorphism of the IL6 gene is associated with elite power performance. J Sci Med Sport. 2010; 13(5):549-553.
35. Eider J, Cieszczyk P, Leonska-Duniec A, Maciejewska A, Sawczuk M, Ficek K, Kotarska K. Association of the 174 G/C polymorphism of the IL6 gene in Polish power-orientated athletes. J Sports Med Phys Fitness. 2013; 53(1):88-92.
36. Ahmetov I I, Gavrilov D N, Astratenkova I V, Druzhevskaya A M, Malinin A V, Romanova E E, Rogozkin V A. The association of ACE, ACTN3 and PPARA gene variants with strength phenotypes in middle school-age children. J Physiol Sci. 2013; 63(1):79-85.
37. Lopez-Leon S, Tuvblad C, Forero D A. Sports genetics: the PPARA gene and athletes' high ability in endurance sports. A systematic review and meta-analysis. Biol Sport. 2016; 33:3-6.
38. Lucia A, Gomez-Gallego F, Barroso I, Rabadán M, Bandrés F, San Juan A F, Chicharro J L, Ekelund U, Brage S, Earnest C P, Wareham N J, Franks P W. PPARGC1A genotype (Gly482Ser) predicts exceptional endurance capacity in European men. J Appl Physiol (1985). 2005; 99(1):344-348.
39. Maciejewska A, Sawczuk M, Cieszczyk P, Mozhayskaya I A, Ahmetov I I. The PPARGC1A gene Gly482Ser in Polish and Russian athletes. J Sports Sci. 2012; 30(1):101-113.
40. Liu X G, Tan L J, Lei S F, Liu Y J, Shen H, Wang L, Yan H, Guo Y F, Xiong D H, Chen X D, Pan F, Yang T L, Zhang Y P, Guo Y, Tang N L, Zhu X Z, Deng H Y, Levy S, Recker R R, Papasian C J, Deng H W. Genome-wide association and replication studies identified TRHR as an important gene for lean body mass. Am J Hum Genet. 2009; 84(3):418-423.
41. Wang P, Ma L H, Wang H Y, Zhang W, Tian Q, Cao D N, Zheng G X, Sun Y L. Association between polymorphisms of vitamin D receptor gene ApaI, BsmI and TaqI and muscular strength in young Chinese women. Int J Sports Med. 2006; 27(3):182-186.
42. Windelinckx A, De Mars G, Beunen G, Aerssens J, Delecluse C, Lefevre J, Thomis M A. Polymorphisms in the vitamin D receptor gene are associated with muscle strength in men and women. Osteoporos Int. 2007; 18(9):1235-1242.
43. Prior S J, Hagberg J M, Paton C M, Douglass L W, Brown M D, McLenithan J C, Roth S M. DNA sequence variation in the promoter region of the VEGF gene impacts VEGF gene expression and maximal oxygen consumption. Am J Physiol Heart Circ Physiol. 2006; 290(5):1848-1855.
44. Ahmetov I I, Khakimullina A M, Popov D V, Missina S S, Vinogradova O L, Rogozkin V A. Polymorphism of the vascular endothelial growth factor gene (VEGF) and aerobic performance in athletes. Hum Physiol. 2008; 34:477-481.
45. Batterham A M, Hopkins W G. A decision tree for controlled trails. Sportsci. 2005; 9:33-39.
46. Egorova E S, Borisova A V, Mustafina L J, Arkhipova A A, Gabbasov R T, Druzhevskaya A M, Astratenkova I V, Ahmetov I I. The polygenic profile of Russian football players. J Sports Sci. 2014; 32(13):1286-93.
47. Calvo M, Rodas G, Vallejo M, Estruch A, Arcas A, Javierre C, Viscor G, Ventura J L. Heritability of explosive power and anaerobic capacity in humans. Eur J Appl Physiol. 2002; 86(3):218-225.
48. Montgomery H E, Marshall R, Hemingway H, Myerson S, Clarkson P, Dollery C, Hayward M, Holliman D E, Jubb M, World M, Thomas E L, Brynes A E, Saeed N, Barnard M, Bell J D, Prasad K, Rayson M, Talmud P J, Humphries S E. Human gene for physical performance. Nature. 1998; 393(6682):221-222.
49. Folland J, Leach B, Little T, Hawker K, Myerson S, Montgomery H, Jones D. Angiotensin-converting enzyme genotype affects the response of human skeletal muscle to functional overload. Exp Physiol. 2000; 85:575-579.
50. Pescatello L S, Kostek M A, Gordish-Dressman H, Thompson P D, Seip R L, Price T B, Angelopoulos T J, Clarkson P M, Gordon P M, Moyna N M, Visich P S, Zoeller R F, Devaney J M, Hoffman E P. ACE ID genotype and the muscle strength and size response to unilateral resistance training. Med Sci Sports Exerc. 2006; 38(6):1074-1081.
51. Pereira A, Costa A M, Izquierdo M, Silva A J, Bastos E, Marques M C. ACE I/D and ACTN3 R/X polymorphisms as potential factors in modulating exercise-related phenotypes in older women in response to a muscle power training stimuli. Age (Dordr). 2013; 35(5):1949-1959.
52. Sukhova Z I, Ivanitskaia V V, Makarova L F, Poluéktova B P, Iazvikov V V. Features of the ultrastructural organization of the muscles of skaters in relation to their sport specialization and muscle fiber composition. Arkh Anat Gistol Embriol. 1985; 89(12):87-90.
53. Petrella J K, Kim J S, Mayhew D L, Cross J M, Bamman M M. Potent myofiber hypertrophy during resistance training in humans is associated with satellite cell-mediated myonuclear addition: a cluster analysis. J Appl Physiol (1985). 2008; 104(6):1736-42.
54. Stone W J, Coulter S P. Strength/endurance effects from three resistance training protocols with women. J Strength Cond Res. 1994; 8:231-234.
55. Häkkinen K, Komi P V, Alen M, Kauhanen H. EMG, muscle fibre and force production characteristics during a 1 year training period in elite weight-lifters. Eur J Appl Physiol Occup Physiol. 1987; 56(4):419-27.

TABLE 1
List of genetic variants analysed

| | | | | Endurance or | |
|---|---|---|---|---|---|
| | | | | power related | |
| Gene | Full name | Functions and associated phenotypes | Polymorphism | allele | References |
| ACE | Angiotensin I | Regulates circulatory homeostasis through the | Alu I/D | Endurance: I | [20, 21] |
| | converting enzyme | synthesis of vasoconstrictor angiotensin II and the | (rs4646994) | Power: D | |
| | | degradation of vasodilator kinins. | | | |
| ACTN3 | α-actinin-3 | Stabilizes the muscle contractile apparatus in fast- | Arg577Ter | Endurance: | [20, 22] |
| | | twitch muscle fibres. | (rs1815739 C/T) | 577Ter (T) | |
| | | | | Power: Arg577 | |
| | | | | (C) | |
| ADRB2 | β-2 adrenoreceptor | Plays a pivotal role in the regulation of the cardiac, | Gly16Arg | Endurance: | [23, 24] |
| | | pulmonary, vascular, endocrine and central nervous | (rs1042713 G/A) | 16Arg (A) | |
| | | system. | Gln27Glu | Endurance: | [25] |
| | | | (rs1042714 C/G) | Gln27 (C) | |
| AGT | Angiotensinogen | Angiotensinogen is an essential component of the | Met235Thr | Power: 235Thr | [26, 27] |
| | | renin-angiotensin system that regulates vascular | (rs699 T/C) | (C) | |
| | | resistance and sodium homeostasis, and thus | | | |
| | | determining blood pressure. | | | |
| BDKRB2 | Bradykinin receptor | Involved in the endothelium-dependent vasodilation. | rs1799722 C/T | Endurance: T | [24] |
| | B2 | | | | |
| COL5A1 | Collagen, type V, α1 | Encodes the pro-α1 chain of type V collagen, the rate- | rs12722 C/T | Endurance: T | [28, 29] |
| | | limiting component of the of type V collagen trimer | (BstUI) | | |
| | | assembly. | | | |
| CRP | C-reactive protein, | Involved in several host defense related functions | rs1205 A/G | Endurance: A | [30, 31] |
| | pentraxin-related | based on its ability to recognize damaged cells and to | | | |
| | | initiate their elimination in the blood. | | | |
| GABPB1 | GA binding protein | Encodes a transcriptional regulator of genes involved | rs7181866 A/G | Endurance: G | [32, 33] |
| (NRF2) | transcription factor, β | in activation of cytochrome oxidase expression and | | | |
| | subunit 1 (nuclear | nuclear control of mitochondrial function. | | | |
| | respiratory factor 2) | | | | |
| IL6 | Interleukin-6 | IL-6 is a pleiotropic cytokine expressed in immune and | −174 C/G | Power: G | [34, 35] |
| | | muscle cells. Involved in a wide variety of biological | (rs1800795) | | |
| | | functions, including regulation of differentiation, | | | |
| | | proliferation and survival of target cells. | | | |
| PPARA | Peroxisome | Regulates liver, heart and skeletal muscle lipid | rs4253778 G/C | Endurance: G | [36, 37] |
| | proliferator-activated | metabolism, glucose homeostasis, mitochondrial | | Power: C | |
| | receptor α | biogenesis, cardiac hypertrophy. | | | |
| PPARGC1A | Peroxisome | Regulates fatty acid oxidation, glucose utilization, | Gly482Ser | Endurance: | [38, 39] |
| | proliferator-activated | mitochondrial biogenesis, thermogenesis, | (rs8192678 G/A) | Gly482 (G) | |
| | receptor γ coactivator | angiogenesis, formation of muscle fibers. | | | |
| | 1 α | | | | |
| TRHR | Thyrotropin-releasing | Stimulates the release of thyroxine, which is important | rs16892496 A/C | Power (muscle | [40] |
| | hormone receptor | in developing skeletal muscle. | | mass): C | |
| VDR | Vitamin D receptor | Involved in sustaining normocalcemia by inhibiting the | Bsml A/G | Power: A | [41, 42] |
| | | production of parathyroid hormone and has effects on | (rs1544410) | | |
| | | bone and skeletal muscle biology. | | | |
| VEGFA | Vascular endothelial | Growth factor active in angiogenesis, vasculogenesis | rs2010963 G/C | Endurance: C | [43, 44] |
| | growth factor A | and endothelial cell growth. | | | |

TABLE 2
Genotype distributions and minor allele frequencies of candidate
genes in athletes of two studies

| | | Genotypes | | | | | | | | |
|---|---|---|---|---|---|---|---|---|---|---|
| Gene and variation | Study | AA | | AB | | BB | | MAF, % | | P_{HW} |
| ACE rs4646994 I/D | S₁ | DD | 10 | ID | 11 | II | 7 | I | 44.6 | 0.2776 |
| | S₂ | | 14 | | 16 | | 9 | | 43.6 | 0.3005 |
| ACTN3 rs1815739 C/T | S₁ | CC | 8 | CT | 10 | TT | 10 | T | 53.6 | 0.1356 |
| | S₂ | | 12 | | 21 | | 6 | | 42.3 | 0.5199 |
| ADRB2 rs1042713 G/A | S₁ | GG | 16 | GA | 10 | AA | 2 | A | 25.0 | 0.8011 |
| | S₂ | | 21 | | 13 | | 5 | | 29.5 | 0.2153 |
| ADRB2 rs1042714 C/G | S₁ | CC | 5 | CG | 15 | GG | 8 | G | 55.4 | 0.6572 |
| | S₂ | | 14 | | 16 | | 9 | | 43.6 | 0.3005 |
| AGT rs699 T/C | S₁ | TT | 9 | TC | 15 | CC | 4 | C | 41.1 | 0.5723 |
| | S₂ | | 17 | | 17 | | 5 | | 34.6 | 0.8171 |
| BDKRB2 rs1799722 C/T | S₁ | CC | 9 | CT | 14 | TT | 5 | T | 42.9 | 0.9122 |
| | S₂ | | 15 | | 17 | | 7 | | 39.7 | 0.5745 |
| COL5A1 rs12722 C/T | S₁ | TT | 8 | TC | 17 | CC | 3 | C | 41.1 | 0.1784 |
| | S₂ | | 13 | | 17 | | 9 | | 44.9 | 0.4576 |
| CRP rs1205 A/G | S₁ | GG | 12 | GA | 12 | AA | 4 | A | 35.7 | 0.7243 |
| | S₂ | | 21 | | 12 | | 6 | | 30.8 | 0.0828 |
| GABPB1 rs7181866 A/G | S₁ | AA | 27 | AG | 1 | GG | 0 | G | 1.8 | 0.9233 |
| | S₂ | | 36 | | 2 | | 1 | | 5.1 | 0.0031* |
| IL6 rs1800795 C/G | S₁ | GG | 10 | GC | 13 | CC | 5 | C | 41.1 | 0.8289 |
| | S₂ | | 17 | | 16 | | 6 | | 35.9 | 0.4977 |
| PPARA rs4253778 G/C | S₁ | GG | 21 | GC | 5 | CC | 2 | C | 16.1 | 0.0736 |
| | S₂ | | 26 | | 11 | | 2 | | 19.2 | 0.5653 |
| PPARGC1A rs8192678 | S₁ | GG | 7 | GA | 18 | AA | 3 | A | 42.9 | 0.0982 |
| G/A | S₂ | | 15 | | 17 | | 7 | | 39.7 | 0.5745 |
| TRHR rs16892496 A/C | S₁ | AA | 14 | AC | 9 | CC | 5 | C | 33.9 | 0.1342 |
| | S₂ | | 15 | | 17 | | 7 | | 39.7 | 0.5745 |
| VDR rs1544410 A/G | S₁ | GG | 11 | GA | 16 | AA | 1 | A | 32.1 | 0.1009 |
| | S₂ | | 16 | | 11 | | 12 | | 44.9 | 0.0073* |
| VEGFA rs2010963 G/C | S₁ | GG | 13 | GC | 11 | CC | 4 | C | 33.9 | 0.5126 |
| | S₂ | | 18 | | 18 | | 3 | | 30.8 | 0.6028 |

MAF—minor allele frequency;
S₁—Study 1;
S₂—Study 2.
*P_{HW} < 0.05 - not consistent with Hardy-Weinberg equilibrium

TABLE 3
Intergroup comparisons of CMJ increases (%) in
response to high- or low-intensity training

| | Increase in CMJ, % | | | | |
|---|---|---|---|---|---|
| | Low- | P₂ | High- | P₂ | |
| | intensity | (paired | intensity | (paired | |
| Group | RT | test) | RT | test) | P₁ |
| Study 1 | | | | | |
| All athletes (n = 28) | 6.4 (5.8) | 0.0009* | 4.1 (8.1) | 0.131 | 0.369 |
| Athletes with P genotype | 3.8 (5.0) | 0.156 | 7.0 (6.7) | 0.125 | 0.429 |
| (n = 11) | | | | | |
| Athletes with E genotype | 8.2 (5.9) | 0.0078* | 2.2 (8.8) | 0.813 | 0.067 |
| (n = 17) | | | | | |
| | P₃ = 0.272 | | P₃ = 0.353 | | |
| Study 2 | | | | | |
| All athletes (n = 39) | 4.6 (4.3) | 0.0056* | 5.0 (4.7) | <0.0001* | 0.932 |
| Athletes with P genotype | 1.0 (4.6) | 0.578 | 7.1 (5.9) | 0.0059* | 0.0046* |
| (n = 17) | | | | | |
| Athletes with E genotype | 7.1 (1.0) | 0.002* | 3.2 (2.5) | 0.0005* | 0.0008* |
| (n = 22) | | | | | |
| | P₃ = 0.0002* | | P₃ = 0.0056* | | |
| Studies 1 and 2 | | | | | |
| All athletes (n = 67) | 5.4 (5.0) | <0.0001* | 4.6 (6.1) | 0.0002* | 0.547 |
| Athletes with P genotype | 2.3 (4.8) | 0.1465 | 7.1 (5.9) | 0.0006* | 0.0052* |
| (n = 28) | | | | | |
| Athletes with E genotype | 7.6 (4.0) | <0.0001* | 2.8 (5.7) | 0.051 | 0.0012* |
| (n = 39) | | | | | |

| | |
|---|---|
| P₃ = 0.0022* | P₃ = 0.0098* |

*P < 0.05 - statistically different values between groups;
P—power;
E—endurance,
RT—resistance training.
P₁ - comparison between athletes with different training types (i.e. low-intensity vs. high-intensity);
P₂ - significant increases in CMJ (paired test);
P₃ - comparison between athletes with different genotype profiles (i.e. power genotype vs. endurance genotype) of the same training modality

TABLE 4
Intergroup comparisons of Aero3 increases (%)
in response to high- or low-intensity training

| | Increase in Aero3, % | | | | |
|---|---|---|---|---|---|
| | Low- | P₂ | High- | P₂ | |
| | intensity | (paired | intensity | (paired | |
| Group | RT | test) | RT | test) | P₁ |
| Study 1 | | | | | |
| All athletes (n = 28) | 2.6 (3.1) | 0.0103* | 4.4 (4.4) | 0.0017* | 0.618 |
| Athletes with P genotype | 2.0 (4.3) | 0.3125 | 6.0 (3.9) | 0.0625 | 0.178 |
| (n = 11) | | | | | |
| Athletes with E genotype | 3.0 (2.2) | 0.0078* | 3.4 (4.6) | 0.0391* | 0.541 |
| (n = 17) | | | | | |
| | P₃ = 0.776 | | P₃ = 0.284 | | |
| Study 2 | | | | | |
| All athletes (n = 39) | 5.8 (3.7) | <0.0001* | 4.2 (3.3) | <0.0001* | 0.218 |
| Athletes with P genotype | 1.7 (0.5) | 0.0156* | 6.8 (2.5) | 0.002* | 0.002* |
| (n = 17) | | | | | |
| Athletes with E genotype | 8.7 (1.6) | 0.002* | 2.1 (2.3) | 0.0161* | <0.0001* |
| (n = 22) | | | | | |
| | P₃ = 0.0001* | | P₃ = 0.002* | | |
| Studies 1 and 2 | | | | | |
| All athletes (n = 67) | 4.3 (3.8) | <0.0001* | 4.3 (3.7) | <0.0001* | 0.711 |
| Athletes with P genotype | 1.8 (2.8) | 0.0171* | 6.5 (2.9) | <0.0001* | 0.0004* |
| (n = 28) | | | | | |
| Athletes with E genotype | 6.0 (3.5) | <0.0001* | 2.6 (3.3) | 0.0004* | 0.0013* |
| (n = 39) | | | | | |

| | |
|---|---|
| P₃ = 0.0004* | P₃ = 0.0026* |

*P < 0.05 - statistically different values between groups;
P—power;
E—endurance,
RT—resistance training.
P₁ - comparison between athletes with different training types (i.e. low-intensity vs. high-intensity);
P₂ - significant increases in Aero3 (paired test);
P₃ - comparison between athletes with different genotype profiles (i.e. power genotype vs. endurance genotype) of the same training modality

TABLE 5
Comparisons of CMJ and Aero3 increases (%) in response to
resistance training between matched and mismatched groups.

| | Group | | | | |
|---|---|---|---|---|---|
| Study | Matched athletes | | Mismatched athletes | | P₃ |
| Study 1 | n = 14 | P₁ | n = 14 | P₂ | |
| | | (paired test) | | (paired test) | |
| Change in CMJ, % | 7.8 (5.9) | 0.0005* | 2.9 (7.2) | 0.175 | 0.0596 |
| Change in Aero3, % | 4.0 (3.1) | 0.0004* | 2.8 (4.3) | 0.0134* | 0.2456 |
| Study 2 | n = 20 | | n = 19 | | |
| Change in CMJ, % | 7.1 (4.1) | <0.0001* | 2.4 (3.5) | 0.0053* | <0.0001* |
| Change in Aero3, % | 7.7 (2.2) | <0.0001* | 1.9 (1.8) | 0.0004* | <0.0001* |
| Studies 1 and 2 | n = 34 | | n = 33 | | |
| Change in CMJ, % | 7.4 (4.9) | <0.0001* | 2.6 (5.3) | 0.0152* | <0.0001* |
| Change in Aero3, % | 6.2 (3.2) | <0.0001* | 2.3 (3.1) | <0.0001* | <0.0001* |

*P₁ and P₂ < 0.05 - significant increases in CMJ and Aero3 (paired test);
*P₃ < 0.05 -significant difference between matched and mismatched groups.
Matched athletes - high-intensity trained with endurance genotype or low-intensity trained with power genotype; mismatched athletes - high-intensity trained with power genotype or low-intensity trained with endurance genotype.

1. A method for predicting whether an individual will respond more to high intensity training or to low intensity training, comprising the step of identifying the allele present at the locus of one or more of the genetic polymorphisms shown in table 1, in a sample obtained from the individual.

2. The method according to claim 1, wherein the method comprises identifying the allele present at the locus of at least two, at least three, at least four or at least five, at least six, at least seven, at least eight, at least nine, at least ten, at least eleven, at least twelve, at least thirteen, at least fourteen or at least all of the polymorphisms shown in table 1.

3. The method according to claim 1 or claim 2, further comprising the step of determining whether the allele identified is an indicator of endurance performance or of power performance.

4. The method according to claim 3, wherein the one or more polymorphisms in the method of the invention comprises or consists of one or more of ACE, ACTN3, CRP, PPARGC1A and VDR.

5. The method according to claim 4, wherein the one or more polymorphisms is selected from:
ACE, ACTN3;
ACE, CRP;
ACE, PPARGC1A;
ACE, VDR;
ACE, ACTN3, CRP;
ACE, ACTN3, PPARGC1A;
ACE, ACTN3, VDR;
ACE, CRP, PPARGC1A,
ACE, CRP, VDR;
ACE, PPARGC1A, VDR;
ACE, ACTN3, CRP, PPARGC1A;
ACE, ACTN3, CRP, VDR;
ACE, ACTN3, CRP, PPARGC1A, VDR;
ACTN3, CRP;
ACTN3, PPARGC1A;
ACTN3, VDR;
ACTN3, CRP, PPARGC1A;
ACTN3, CRP, VDR;
ACTN3, PPARGC1A, VDR;
CRP, PPARGC1A;
CRP, VDR;
CRP, PPARGC1A, VDR; and
PPARGC1A, VDR.

6. The method according to claim 4 or claim 5, wherein the allele is a power allele.

7. The method according to claim 3, wherein the one or more polymorphisms in the method of the invention comprises or consists of one or more of one or more of ACE, ACTN3, CRP, and VDR.

8. The method according to claim 7, wherein the one or more polymorphisms may comprise or consist of any of the following combinations of polymorphisms:
ACE, ACTN3;
ACE, CRP;
ACE, VDR;
ACE, ACTN3, CRP;
ACE, ACTN3, VDR;
ACE, CRP, VDR;
ACE, ACTN3, CRP, VDR;
ACTN3, CRP;
ACTN3, VDR;
ACTN3, CRP, VDR; and
CRP, VDR.

9. The method according to claim 7 or 8, wherein the allele is an endurance allele.

10. The method according to any preceding claim, wherein the one or more polymorphism further comprises any one or more of TRHR, PPARA and IL6.

11. The method of any preceding claim, comprising the step of weighting of one or more of the polymorphisms.

12. The method of claim 11, comprising the step of weighting the allele at any one or more of ACE, ACTN3, CRP, PPARGC1A and VDR more heavily than an allele at another locus.

13. The method of claim 12, wherein the allele at one or more of ACE, ACTN3, CRP, PPARGC1A and VDR is weighted by a factor of about 2 when compared with an allele at another locus.

14. The method according to any preceding claim, comprising the step of testing the aerobic fitness or explosive power of the individual before and/or after training.

15. The method according to any preceding claim for improving training and/or for increasing fitness of an individual.

16. Use of the results obtainable or obtained by a method according to any preceding claim for improving training and/or for increasing fitness of an individual.
